(19) United States
(12) Patent Application Publication
(10) Pub. No.: US 2023/0277119 A1
(43) Pub. Date: Sep. 7, 2023

(54) METHOD AND SYSTEM FOR DETECTING SKIN CONTACT REACTIONS AND MAKING ASSOCIATED SAFE PRODUCT RECOMMENDATIONS

(71) Applicant: All Skin Inc, Calgary (CA)

(72) Inventors: Ryan T. Lewinson, Calgary (CA); Isabelle Vallerand, Calgary (CA); John-Douglas Matthew Hughes, Calgary (CA)

(73) Assignee: All Skin Inc, Calgary (CA)

(21) Appl. No.: 18/040,762

(22) PCT Filed: Aug. 3, 2021
(86) PCT No.: PCT/CA2021/051082

§ 371 Date: Feb. 6, 2023

Related U.S. Application Data
(60) Provisional application No. 63/067,256, filed on Aug. 18, 2020.

(51) Int. Cl. A61B 5/00

(52) U.S. Cl. CPC A61B 5/442; A61B 5/0077; G16H 50/20

(57) ABSTRACT

We provide a system that detects skin contact reactions to a set of allergens. During operation, the system enables a user to acquire an image of a patch area through a camera in a portable device, wherein the patch area is located on the skin of a patient, and wherein a patch containing the set of allergens was applied to the patch area for a period of time and removed before the image was acquired. Next, the system preprocesses the image so that a resulting preprocessed image of the patch area has a prespecified size and orientation. The system then performs image-processing operations on the preprocessed image to identify regions of the patch area corresponding to positive skin contact reactions. The system labels each identified region with a specific allergen that was applied to the region to produce test results, which identify specific allergens that produced positive skin contact reactions.

### RELATED APPLICATION

This application claims priority under 35 U.S.C. § 119 to U.S. Provisional Application No. 63/067,256, entitled “Detection and Labelling of Skin Contact Reactions and Recommender System for Safe Product List” by inventors Ryan T. Lewinson, et al., filed on 18 Aug. 2020, the contents of which are incorporated by reference herein.

### BACKGROUND

### Field

The disclosed embodiments generally relate to techniques for detecting skin allergy reactions. More specifically, the disclosed embodiments provide a system for detecting and labelling skin contact reactions and using this information to make associated safe product recommendations.

### Related Art

Approximately 20% of the general population experience rashes caused by skin allergens, a condition known as allergic contact dermatitis (ACD). ACD is a chronic condition that produces erythematous (red), pruritic (itchy), painful, eczematous rashes, sometimes also associated with skin blisters or fissures (cracks). ACD is the result of a delayed hypersensitivity reaction to an allergen, which typically occurs 48 to 72 hours after exposure. These allergens may be found in the workplace, or in household, cosmetic, or personal hygiene products. ACD frequently affects the face, especially the eyelids, along with the hands and feet; however, ACD can occur anywhere on the body as a result of allergen exposure. The most common allergens implicated in the development of ACD include metals (e.g. nickel, cobalt), fragrances, preservatives (e.g. methylisothiazolinone/methylchloroisothiazolinone, formaldehyde), topical antibiotics (e.g. neomycin, bacitracin) and dyes (e.g. p-phenylenediamine). ACD is a chronic disease with no cure; however, it can be avoided, once the offending allergen is identified and removed from use.

Currently, the gold standard for diagnosing ACD is “patch testing,” a procedure which has been in existence for over 100 years. Patch testing entails having a series of patches which contain an individual allergen suspended in petrolatum gel (or another medium) applied to the skin, typically on the upper back, for approximately 48 hours. The results of this exposure (delayed hypersensitivity reaction) are assessed after an additional 48 to 72 hours. A map of the applied patches is then used as a reference to determine an individual's specific allergens.

Although patch testing has been used for many years, it has its disadvantages. For instance, patch testing is typically only offered by dermatologists with expertise or special interest in ACD, and these dermatologists typically only practice in large urban centers. As such, access to care is quite limited for patients who live in rural areas, and wait times for all patients often exceed one year. During these long wait times, patients suffer. Skin symptoms, visual changes in appearance, and anxiety regarding their personal care products exacerbate patient morbidity. High operating costs, and costs to the patient, are likely aggravated by routinely screening each patient for a large number of allergens (usually >100), which inevitably includes allergens that are very rarely found in common commercial products. Moreover, the overall time required to administer the patch testing procedure renders it largely prohibitive for most dermatologists to offer the procedure amidst a busy practice. The treatment of ACD ultimately necessitates the identification and elimination of causative allergens. However, limited access to health care providers, long waitlists, geographic restrictions, and exorbitant costs all contribute to decreased access to care and increased patient morbidity.

Artificial-intelligence-based decision-making has grown rapidly in the field of dermatology and skin care, with previous use cases being developed for detection of melanoma skin cancer, skin feature tracking over time, and beauty advice. While these modern technologies have not yet been applied toward patch testing, they offer significant opportunity to expand ACD testing by allowing patients to safely evaluate reactions remotely, without physically seeing a skilled evaluator such as a dermatologist.

Hence, what is needed is a new technique for diagnosing ACD and identifying associated allergens without the shortcomings of existing patch testing techniques.

### SUMMARY

The disclosed embodiments relate to a system that detects skin contact reactions to a set of allergens. During operation, the system enables a user to acquire an image of a patch area through a camera in a portable device, wherein the patch area is located on the skin of a patient, and wherein a patch containing the set of allergens was applied to the patch area for a period of time and removed before the image was acquired. Next, the system preprocesses the image so that a resulting preprocessed image of the patch area has a prespecified size and orientation. The system then performs image-processing operations on the preprocessed image to identify regions of the patch area corresponding to positive skin contact reactions. Finally, the system labels each identified region with a specific allergen that was applied to the region to produce test results, which identify specific allergens that produced positive skin contact reactions.

In some embodiments, the patch was applied to the patch area for approximately two days and was removed for approximately two to three days before the image was acquired.

In some embodiments, while enabling the user to acquire the image through the camera, the system displays an image of what is being viewed by the camera to a user, and allows the user to adjust a position of the camera to acquire a visible image of the entire patch area.

In some embodiments, more than one image is acquired and processed to account for differing lighting conditions, skin tone, skin pigment, and skin textural changes.

In some embodiments, multiple images are acquired at different times. For example, a first image can be acquired at 48 hours, and a second image can be acquired at 72 hours. This enables us to assess for irritant versus allergic reactions based on the difference between images/time points, and also to possibly use both images for calibration of orientation/registration markings based on background skin and other identified features of the patch/markings.

In some embodiments, preprocessing the image involves performing one or more of the following preprocessing operations: shifting the image; rotating the image; scaling the image; cropping the image; adjusting the image for baseline skin tone or color; using machine learning to enhance or normalize selected qualities of the image; and adjusting a brightness and/or a contrast of the image.

In some embodiments, preprocessing the image includes using a machine-learning technique to normalize the image for varying light conditions, skin tone, and contrast.

In some embodiments, one or more of the preprocessing operations are performed with reference to patch registration marks, which were transferred from the patch to the skin while the patch was applied to the skin.

In some embodiments, while performing the image-processing operations to identify the regions of the patch area corresponding to positive skin contact reactions, the system uses a machine-learning model to recognize positive skin contact reactions based on variations in skin color and/or skin texture.

In some embodiments, the machine-learning model comprises a neural network or related deep-learning architecture.

In some embodiments, the system additionally uses the test results to filter a product catalog based on lists of known product ingredients to produce a set of safe product recommendations for the patient.

In some embodiments, while producing the set of safe product recommendations, the system additionally considers patient data and preferences, which can include: patient age, patient gender, patient skin type and texture, pre-existing skin conditions of the patient, climate-related aspects of the patient's variable geographic location/selected geographic location, and product preferences of the patient.

In some embodiments, the set of safe product recommendations produce a personalized list further tailored and/or filtered to an individual retail vendor or brand.

In some embodiments, the preprocessing operations and the image-processing operations are performed by one or more of: the portable device, and a remote cloud-computing system.

In some embodiments, the portable device comprises one of: a smartphone, a tablet computer and a digital camera with a processor.

The disclosed embodiments also relate to a patch that facilitates testing a set of allergens for skin contact reactions. This patch includes a set of wells, which are configured to hold the set of allergens. It also includes adhesive sections located on the surface of the patch for bonding the patch to a patient's skin. Finally, the patch includes registration marks comprised of dye or other temporary skin coloring mechanism infused into the patch, wherein the registrations marks are transferred to the skin of the patient when the patch is applied to the patient. (Note that the registration markings either appear on the patch itself, or on the patient's skin as a residual after the patch is applied.)

In some embodiments, the patch further includes a water-resistant coating.

In some embodiments, the patch further includes a backing, which is configured to be peeled away to expose the adhesive sections and the registration marks before the patch is applied to the patient.

In some embodiments, the set of wells in the patch are shipped pre-populated with the set of allergens.

In some embodiments, a user loads the set of allergens into the set of wells before the patch is applied to the patient.

In some embodiments, patch orientation registration markings are drawn on the skin by the patient or other observer, which may involve either tracing the periphery of the patch, or by tracing within a pre-cut registration template.

In some embodiments, patch and allergen orientation is determined from a prior photo taken of the patient wearing the patch, which may include goniometer based measurements from the portable device, QR-code readings from the patch surface, or manual labelling by the patient or observer.

In some embodiments, patch orientation is discerned from a variety of different well shapes and arrangements within the patch.

In some embodiments, patch orientation is discerned from markings on a secondary adhesive layer that remains on the skin for a period of time after the patch is removed.

In some embodiments, patch orientation is determined manually.

### BRIEF DESCRIPTION OF THE FIGURES

FIGS. 1A-1B presents a detail flow chart illustrating an exemplary process of detecting and labelling skin contact reactions and using this information to make associated safe product recommendations in accordance with the disclosed embodiments.

FIGS. 2A-2B presents a flow diagram illustrating an exemplary process for how an image of a patch test result can be used to identify allergens that cause skin contact reactions in accordance with the disclosed embodiments.

FIG. 3 illustrates an exemplary product recommendation system in accordance with the disclosed embodiments.

FIG. 4 illustrates an exemplary patch with multiple wells for different allergens and associated registration marks in accordance with the disclosed embodiments.

FIG. 5 presents a high-level flow chart illustrating the process of detecting skin contact reactions in accordance with the disclosed embodiments.

### DETAILED DESCRIPTION

The following description is presented to enable any person skilled in the art to make and use the present embodiments, and is provided in the context of a particular application and its requirements. Various modifications to the disclosed embodiments will be readily apparent to those skilled in the art, and the general principles defined herein may be applied to other embodiments and applications without departing from the spirit and scope of the present embodiments. Thus, the present embodiments are not limited to the embodiments shown, but are to be accorded the widest scope consistent with the principles and features disclosed herein.

The data structures and code described in this detailed description are typically stored on a computer-readable storage medium, which may be any device or medium that can store code and/or data for use by a computer system. The computer-readable storage medium includes, but is not limited to, volatile memory, non-volatile memory, magnetic and optical storage devices such as disk drives, magnetic tape, CDs (compact discs), DVDs (digital versatile discs or digital video discs), or other media capable of storing computer-readable media now known or later developed.

The methods and processes described in the detailed description section can be embodied as code and/or data, which can be stored in a computer-readable storage medium as described above. When a computer system reads and executes the code and/or data stored on the computer-readable storage medium, the computer system performs the methods and processes embodied as data structures and code and stored within the computer-readable storage medium. Furthermore, the methods and processes described below can be included in hardware modules. For example, the hardware modules can include, but are not limited to, application-specific integrated circuit (ASIC) chips, field-programmable gate arrays (FPGAs), and other programmable-logic devices now known or later developed. When the hardware modules are activated, the hardware modules perform the methods and processes included within the hardware modules.

### Discussion

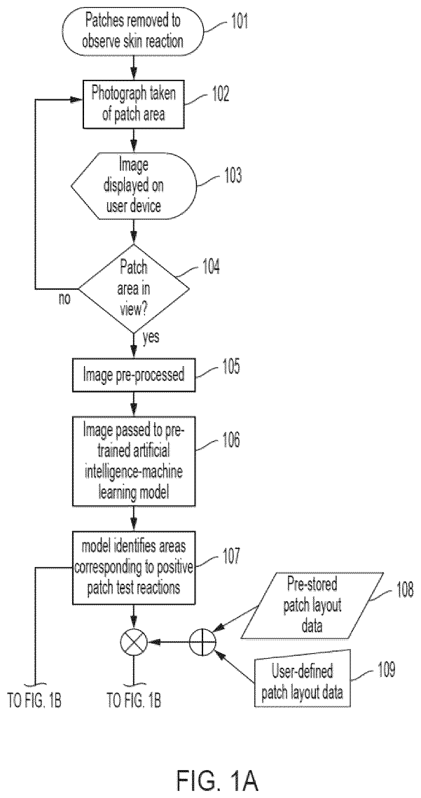

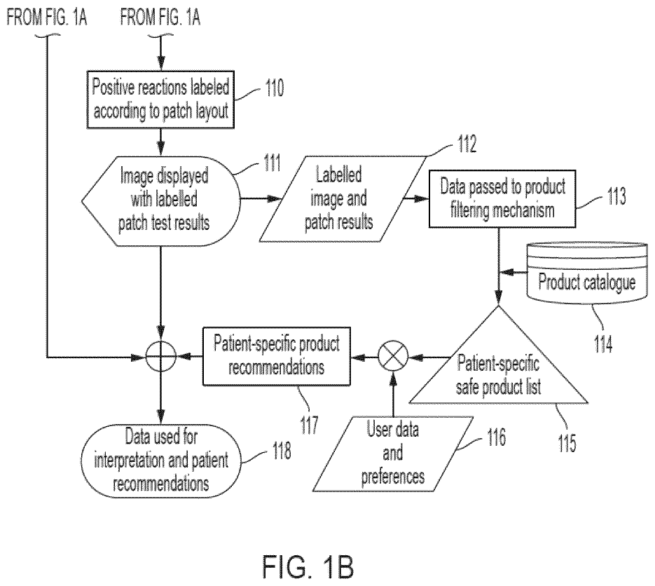

The present invention relates to a computer-assisted method for diagnosing and labelling skin contact allergen and irritant reactions, and using this information to make associated safe product recommendations. FIGS. 1A and 1B present a flow chart of the general approach. Referring to FIG. 1A, step 101 involves the removal of the patch test from the skin. A typical patch testing procedure involves applying an array of varying concentrations of common allergens and irritants to the skin, and leaving this in place for a period of time (˜48 hrs). This array may be standard issue or customized. Time is provided to allow the applied chemicals sufficient skin exposure so as to induce a skin reaction in susceptible individuals. Importantly, this reaction is safe and non-life-threatening. After removal of the patch, an additional period of time (˜48 to 72 hrs) is provided before progressing to the next step.

Step 102 involves the collection of a photograph of the full patch test area (or area to be analyzed). This may be accomplished with a handheld smart device, a personal camera, or any other photographic device that transfers the image to an electronic medium. This image is then displayed to the user in step 103.

Step 104 determines whether the full area to be assessed is within view on the image. This can be done manually through user determination, and it can alternatively be done electronically by way of a computer-assisted determination of image quality. If the image quality is insufficient, the user is prompted to provide a new image; otherwise, the technique proceeds.

Step 105 involves image pre-processing, which may or may not be required depending on the image source and the image quality. These processing steps may include, but are not limited to, image cropping, image translation, image rotation, and image brightness and contrast adjustments.

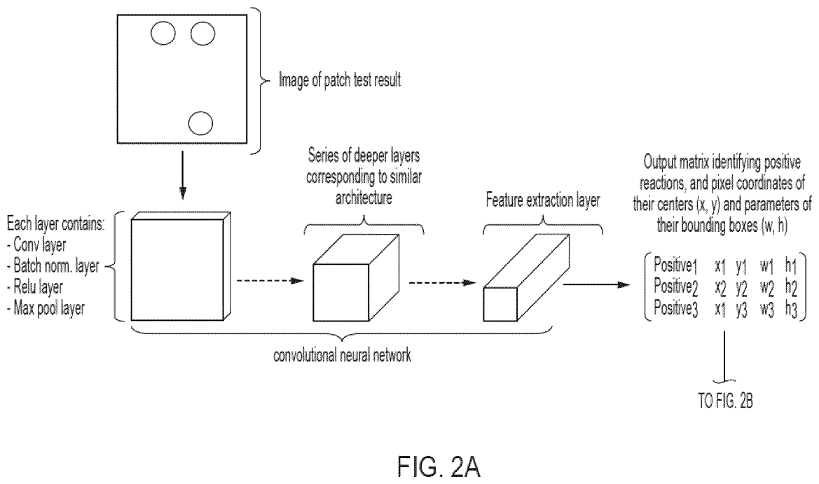

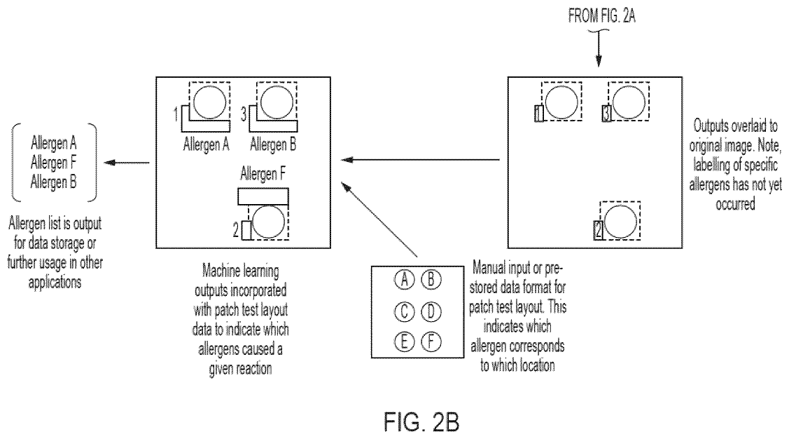

Step 106 passes the patch test image into a pre-trained artificial intelligence/machine-learning model, which is used to identify areas corresponding to positive reactions in step 107. This machine-learning model can be implemented using any one of a number of convolutional neural networks, feed-forward neural networks, object-detection systems, or other machine-learning models. By using an appropriately trained machine-learning model, the system is able to account for variations in image quality, patch orientation, reaction severity, baseline skin color, baseline skin texture, and other complexities and/or variables while offering a rapid solution for analytics once deployed. In the disclosed embodiment, our system implements a modified You Only Look Once v2 (YOLOv2) technique, wherein the image is passed through a series of layers with convolutions, batch normalization, rectified linear unit activation and max pooling, wherein feature weights are learned. With a trained model, features can be extracted from a given layer to facilitate final object detection. In this way, objects (i.e., positive reactions) can be detected and denoted by a bounding box and the associated coordinates in relation to the original image can be saved. This process is illustrated in the diagram that appears in FIGS. 2A and 2B. This information can be used in step 118 to facilitate data interpretation and associated patient recommendation if the user decides not to make use of the operations performed in steps 112-117.

Data sets 108 and 109 provide supplemental information, which identifies the specific chemicals that were tested, and the specific patch layout for the chemicals. The user can input this information manually, or the information can be automatically imported from data sets 108 and 109. In one embodiment, a QR code associated with the patch applied can be scanned to import this data. In another embodiment, a list of predefined patch layouts can be displayed and selected by the user.

Step 110 integrates data from the machine-learning output in step 107 and the supplemental patch layout information from data sets 108 and 109 to label positive reactions according to the chemical corresponding to that reaction position. This information is then displayed to the user in step 111 as the original image with a bounding box overlay corresponding to positive patch results, which can then be labelled according to the specific contact allergens or irritants causing the positive reactions (see FIGS. 2A and 2B).

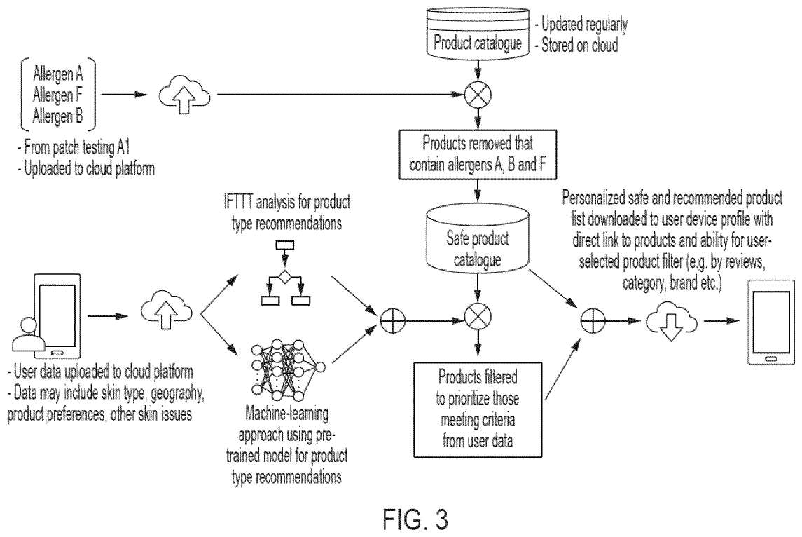

Note that step 118 can be initiated directly in the case where the user wishes to interpret this information manually. However, as illustrated in boxes 112-117, our invention also provides a technique for integrating patch test results data with external data, such as product information from a product catalog 114 and user data and user preferences from data set 116 to provide product recommendations. (See FIG. 3 for more details.) This can be important because some chemicals used in patch testing that appear in products are unfamiliar to the general public and to clinicians who are inexperienced in patch testing.

The labelled image and patch test results from data set 112 (i.e., the allergens and irritants causing positive reactions) are exported from the machine-learning system as saved data. Depending on the platform used to initiate this process, the data can be saved locally on a user smart device, locally on a computer system, or on a remote cloud-based platform.

Data are then passed to a product filtering mechanism in step 113, which incorporates external data from product catalog 114. Product catalog 114 comprises a list of products (personal hygiene, cosmetic, cosmeceutical, general purpose/cleaning, occupational), which are available on the market, as well as common products known to cause contact dermatitis (e.g., detergents, household cleaners, etc.). Product catalog 114 includes data corresponding to, but not limited to, full ingredient lists (including allergens and irritants), the product use intention (for example, moisturizers, sunscreens, shampoos, detergents, etc.), customer ratings, and pricing. By incorporating known product ingredient lists from product catalog 114 with data from patch testing results, the data in product catalog 114 can be filtered in step 115 to remove all products that contain allergens and irritants that caused a positive reaction. Because new products are released to the market frequently, this list will be updated in real time.

Data set 116 contains available user data and preferences. For example, this data may comprise, but is not limited to information about: patient age and gender; skin type; pigment and texture (such as oily/dry etc.), which is obtained through user input or through image classification; geography (e.g. relating to climate, temperature, humidity etc.); other skin issues; and other products used. By incorporating this data with the data from safe product list 115, a user-specific product recommendation can be generated, which excludes products containing chemicals that induced positive patch test reactions.

This user-specific product recommendation can be produced using a simple “if this, then that” (IFTTT) technique, or may instead use a machine-learning approach, wherein feature weights are learned by training the recommendation system to produce the desired outputs. A clustering model can also be developed to identify groups of individuals with data similarities. These “smart” approaches, which use machine learning and clustering analyses, can identify correlations that are not readily apparent to help deal with multivariable complexity.

For example, a 27-year-old female with Fitzpatrick skin phototype II (fair skin) and a history of atopic dermatitis (eczema) who lives in Calgary, Canada (typically cold and dry), might be recommended, by way of an IFTTT technique or machine-learning technique, to use a sunscreen-containing (given her lighter skin phototype) moisturizing cream (instead of a lighter lotion, given the cold dry environment and history of atopic dermatitis) that excludes any of the contact allergens and irritants that she may have tested positive for, and the technique can also provide a list of products that meet these criteria. By using a machine-learning approach, it may also be determined that individuals with similar geography, age, gender and/or other variables tend to have higher personal preference scores for a particular product type, which can help to guide product recommendations.

Hence, our system: (1) enables a user to perform a patch test in a home or clinical environment; (2) identifies positive allergens or irritants by way of artificial intelligence; and (3) provides a computer-aided smart recommendation for products, which not only excludes allergens or irritants that tested positive in the patch test, but also incorporates user data and personal preferences to provide guidance on product selection to optimize skin care.

### Exemplary Applications

Our system can be used via a consumer-grade patch, which is prefabricated with common allergens for patch testing. This patch can be applied to the user outside of a medical facility and the patch test allergen layout can be determined via a QR code or in-app product selector. Next, an image can be acquired by the user with their smart device that has a camera on the day the patch test result is to be interpreted, and the image can be passed to a cloud-based network, which applies a pre-trained machine-learning model to the image to identify skin contact reactions. By incorporating the identified skin contact reactions with the defined patch test layout, our system identifies and labels the allergens causing positive skin reactions.

This data is then passed to a recommendation system that filters a library of products based on associated ingredient lists and removes products that contain the positive allergens. If the user has provided additional personal data via a user profile, this personal data is used to refine the safe product list by means of machine learning or an IFTTT technique to create a customized list of recommended products, which are safe and free of any contact allergens the individual tested positive to, and are congruent with their skin type and personal information and preferences. This list of recommended products can be fed back to the user's device where this information is stored in an associated user profile, with direct product links to facilitate purchases from the recommended products list, which can be accessed and revisited over time.

In another example, the above procedure is repeated, but with user data and patch test result data that are used by a machine-learning system or other technique to facilitate the manufacture of a customized skin care product for the user.

In yet another example, an open application test is performed, whereby a user suspends a household product in petrolatum (petroleum jelly) and applies a patch containing the petrolatum to the skin. Our system is then used to determine if a positive reaction occurred. Next, by using the product catalogue, the tested product can be cross-referenced (either manually or via an automated computer-based technique) to identify likely problematic allergens, and associated products.

In an additional example, the system is used by a rural/remote health care provider at a location where access to patch testing may be limited. Prefabricated patch tests with common allergens are applied to the patient and then on a return visit, the physician can use our system to store the image and associated positive reactions in the patient's electronic medical record for later access and referral purposes, and to generate a safe product list for the patient. In this way, our system facilitates initial diagnosis and management of allergic contact dermatitis while the patient awaits referral to a specialized center, if required, for further testing of additional allergens at a later date.

In further example, the system can be used in a specialized dermatology center that offers patch testing, whereby the technology is used to document all positive reactions and rapidly identify the causative allergen based on a user-defined patch test layout. These data can then be exported to the patient's electronic medical record for later reference, and an associated custom product list can be generated. This allows the patch testing analysis procedure to be expedited via computer-aided technology, rather than manually documenting over 60 individual patch test results, and then manually sorting and arranging a safe products list, thereby allowing more patients to be seen within a given time period.

### Patch Design

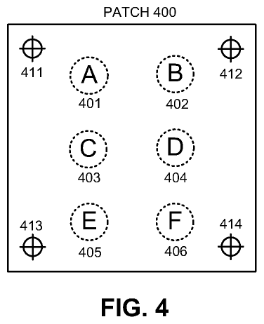

FIG. 4 illustrates an exemplary patch 400 that facilitates testing a set of allergens for skin contact reactions in accordance with the disclosed embodiments. This patch 400 includes a set of wells 401-406, which are configured to hold the set of allergens (A, B, C, D, E and F). Note that the set of wells 401-406 can be shipped pre-populated with the set of allergens. Alternatively, a user can load the set of allergens into the set of wells 401-406 before the patch is applied to the patient. The patch also includes a set of registration marks 411-414, which are comprised of a dye, such as henna, or a temporary tattoo, which is infused into the patch so that the registrations marks 411-414 are transferred to the skin of the patient when the patch is applied to the patient. The patch can also include a water-resistant coating (not shown) to enable the patient to shower when wearing the patch. The patch can additionally include a backing (not shown), which is configured to be peeled away to expose the adhesive sections and the registration marks before the patch is applied to the patient. As noted in the summary section above, there exist a number of other alternative approaches for registering the patch layout.

### Detecting Skin Contact Reactions

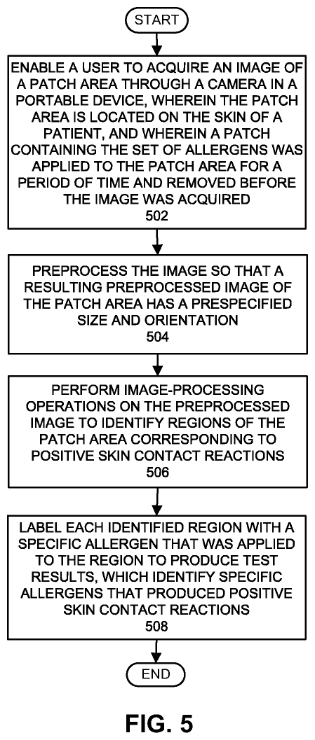

FIG. 5 presents a high-level flow chart illustrating the process of detecting skin contact reactions in accordance with the disclosed embodiments. During operation, the system enables a user to acquire an image of a patch area through a camera in a portable device, wherein the patch area is located on the skin of a patient, and wherein a patch containing the set of allergens was applied to the patch area for a period of time and removed before the image was acquired (step 502). Next, the system preprocesses the image so that a resulting preprocessed image of the patch area has a prespecified size and orientation (step 504). The system then performs image-processing operations on the preprocessed image to identify regions of the patch area corresponding to positive skin contact reactions (step 506). Finally, the system labels each identified region with a specific allergen that was applied to the region to produce test results, which identify specific allergens that produced positive skin contact reactions (step 506).

Various modifications to the disclosed embodiments will be readily apparent to those skilled in the art, and the general principles defined herein may be applied to other embodiments and applications without departing from the spirit and scope of the present invention. Thus, the present invention is not limited to the embodiments shown, but is to be accorded the widest scope consistent with the principles and features disclosed herein.

The foregoing descriptions of embodiments have been presented for purposes of illustration and description only. They are not intended to be exhaustive or to limit the present description to the forms disclosed. Accordingly, many modifications and variations will be apparent to practitioners skilled in the art. Additionally, the above disclosure is not intended to limit the present description. The scope of the present description is defined by the appended claims.

What is claimed is:

1. A method for detecting skin contact reactions to a set of allergens, comprising:
enabling a user to acquire an image of a patch area through a camera in a portable device, wherein the patch area is located on the skin of a patient, and wherein a patch containing the set of allergens was applied to the patch area for a period of time and removed before the image was acquired;
preprocessing the image so that a resulting preprocessed image of the patch area has a prespecified size and orientation;
performing image-processing operations on the raw or preprocessed image to identify regions of the patch area corresponding to positive skin contact reactions; and
labelling each identified region with a specific allergen that was applied to the region to produce test results, which identify specific allergens that produced positive skin contact reactions.

2. The method of claim 1, wherein the patch was applied to the patch area for approximately two days and was removed for approximately two to three days before the image was acquired.

3. The method of claim 1, wherein enabling the user to acquire the image through the camera involves:
displaying an image of what is being viewed by the camera to a user; and
allowing the user to adjust a position of the camera to acquire a visible image of the entire patch area.

4. The method of claim 1, wherein more than one image is acquired and processed to account for differing lighting conditions, skin tone, and skin textural changes.

5. The method of claim 1, wherein multiple images are acquired at different times.

6. The method of claim 1, wherein preprocessing the image involves performing one or more of the following preprocessing operations:
shifting the image;
rotating the image;
scaling the image;
cropping the image;
adjusting a brightness of the image;
adjusting a contrast of the image;
adjusting the image for baseline skin tone, pigment or color; and
using machine learning to enhance or normalize selected qualities of the image.

7. The method of claim 6, wherein one or more of the preprocessing operations are performed with reference to patch registration marks, which were transferred from the patch to the skin while the patch was applied to the skin.

8. The method of claim 1, wherein preprocessing the image includes using a machine-learning technique to normalize the image for varying light conditions, skin tone, and contrast.

9. The method of claim 1, wherein performing the image-processing operations to identify the regions of the patch area corresponding to positive skin contact reactions comprises using a machine-learning model to recognize positive skin contact reactions based on variations in skin color/pigment and/or skin texture.

10. The method of claim 8, wherein the machine-learning model comprises a neural network or a related deep-learning architecture.

11. The method of claim 1, wherein the method further comprises using the test results to filter a product catalog based on lists of known product ingredients to produce a set of safe product recommendations for the patient.

12. The method of claim 11, wherein producing the set of safe product recommendations additionally involves considering patient data and preferences, which can include: patient age, patient gender, patient skin type and texture, pre-existing skin concerns, climate-related aspects of the patient's current or selected geographic location, and product preferences of the patient.

13. The method of claim 12, wherein the set of safe product recommendations produce a personalized list further tailored and/or filtered to an individual retail vendor or brand.

14. The method of claim 1, wherein the preprocessing operations and the image-processing operations are performed by one or more of: the portable device; and a remote cloud-computing system.

15. The method of claim 1, wherein the portable device comprises one of: a smartphone, a tablet computer and a digital camera with a processor.

16. A system for detecting skin contact reactions to a set of allergens, comprising:
a portable device including a camera, which is configured to enable a user to acquire an image of a patch area, wherein the patch area is located on the skin of a patient, and wherein a patch containing the set of allergens was applied to the patch area for a period of time and removed before the image was acquired; and
a processing mechanism, which is configured to,
preprocess the image so that a resulting preprocessed image of the patch area has a prespecified size and orientation,
perform image-processing operations on the preprocessed image to identify regions of the patch area corresponding to positive skin contact reactions, and
label each identified region with a specific allergen that was applied to the region to produce test results, which identify specific allergens that produced positive skin contact reactions.

17. The system of claim 16, wherein the patch was applied to the patch area for approximately two days and was removed for approximately two to three days before the image was acquired.

18. The system of claim 16, wherein while enabling the user to acquire the image through the camera, the system:
displays an image of what is being viewed by the camera to a user; and
allows the user to adjust a position of the camera to acquire a visible image of the entire patch area.

19. The system of claim 16, wherein more than one image is acquired and processed to account for differing lighting conditions, skin tone, and skin textural changes.

20. The system of claim 19, wherein multiple images are acquired at different times.

21. The system of claim 16, wherein while preprocessing the image, the system performs one or more of the following preprocessing operations:
shifting the image;
rotating the image;
scaling the image;
cropping the image;
adjusting a brightness of the image;
adjusting a contrast of the image;
adjusting the image for baseline skin tone or color; and
using machine learning to enhance or normalize selected qualities of the image.

22. The system of claim 21, wherein one or more of the preprocessing operations are performed with reference to patch registration marks, which were transferred from the patch to the skin while the patch was applied to the skin.

23. The system of claim 16, wherein while preprocessing the image, the system uses a machine-learning technique to normalize the image for varying light conditions, skin tone, and contrast.

24. The system of claim 16, wherein while performing the image-processing operations to identify the regions of the patch area corresponding to positive skin contact reactions, the system uses a machine-learning model to recognize positive skin contact reactions based on variations in skin color and/or skin texture.

25. The system of claim 24, wherein the machine-learning model comprises a neural network or a related deep-learning architecture.

26. The system of claim 16, wherein the processing mechanism additionally uses the test results to filter a product catalog based on lists of known product ingredients to produce a set of safe product recommendations for the patient.

27. The system of claim 26, wherein while producing the set of safe product recommendations, the processing mechanism additionally considers patient data and preferences, which can include: patient age, patient gender, patient skin type and texture, pre-existing skin concerns, climate-related aspects of the patient's current or selected geographic location, and product preferences of the patient.

28. The system of claim 27, wherein the set of safe product recommendations produce a personalized list further tailored and/or filtered to an individual retail vendor or brand.

29. The system of claim 16, wherein the processing mechanism is located on one or more of: the portable device; and a remote cloud-computing system.

30. The method of claim 16, wherein the portable device comprises one of: a smartphone, a tablet computer and a digital camera with a processor.

31. A patch that facilitates testing a set of allergens for skin contact reactions, comprising:
the patch, which includes a set of wells configured to hold the set of allergens;
adhesive sections located on the patch for bonding the patch to a patient's skin; and
registration marks, which are transferred to the skin of the patient when the patch is applied to the patient.

32. The patch of claim 31, wherein the registration marks are comprised of dye or temporary tattoo infused into the patch.

33. The patch of claim 31, wherein the registration marks are comprised of a secondary adhesive layer that remains on the skin for a period of time after the patch is removed.

34. The patch of claim 31, wherein the patch further includes a water-resistant coating.

35. The patch of claim 31, wherein the patch further includes a backing, which is configured to be peeled away to expose the adhesive sections and the registration marks before the patch is applied to the patient.

36. The patch of claim 31, wherein the set of wells in the patch are shipped pre-populated with the set of allergens.

37. The patch of claim 31, wherein a user loads the set of allergens into the set of wells before the patch is applied to the patient.
